SEQUENCE LISTING (18 sequences)

SEQ ID NO: 1 - DNA, 24 bp, Artificial
  synthetic oligonucleotide
tgcagccaaagtgaagagggaaga

SEQ ID NO: 2 - DNA, 24 bp, Artificial
  synthetic oligonucleotide
catagcgagcagcccaaagaagaa

SEQ ID NO: 3 - DNA, 20 bp, Artificial
  synthetic oligonucleotide
actgtcaacttcggggacac

SEQ ID NO: 4 - DNA, 20 bp, Artificial
  synthetic oligonucleotide
catgcctaaacgcttcatca

SEQ ID NO: 5 - DNA, 20 bp, Artificial
  synthetic oligonucleotide
tcatcgtctgctcctcctct

SEQ ID NO: 6 - DNA, 20 bp, Artificial
  synthetic oligonucleotide
aggtgtcatcagcagccttt

SEQ ID NO: 7 - DNA, 20 bp, Artificial
  synthetic oligonucleotide
caatcaggtggtggtgtcag

SEQ ID NO: 8 - DNA, 20 bp, Artificial
  synthetic oligonucleotide
gctcctggactgttttctgc

SEQ ID NO: 9 - DNA, 21 bp, Artificial
  synthetic oligonucleotide
aagtcgcctcgaagatacaca

SEQ ID NO: 10 - DNA, 21 bp, Artificial
  synthetic oligonucleotide
aaggagagaacactgctcgtg

SEQ ID NO: 11 - DNA, 20 bp, Artificial
  synthetic oligonucleotide
ggacagagacgacaagcaca

SEQ ID NO: 12 - DNA, 20 bp, Artificial
  synthetic oligonucleotide
catctgtgtagggcatgtgg

SEQ ID NO: 13 - DNA, 23 bp, Artificial
  synthetic oligonucleotide
tacaccactctggcagccacact

SEQ ID NO: 14 - DNA, 24 bp, Artificial
  synthetic oligonucleotide
cggtgggtacattggtgacagaac

SEQ ID NO: 15 - DNA, 20 bp, Artificial
  synthetic oligonucleotide
gcaccgtcaaggctgagaac

SEQ ID NO: 16 - DNA, 20 bp, Artificial
  synthetic oligonucleotide
atggtggtgaagacgccagt

SEQ ID NO: 17 - DNA, 25 bp, Artificial
  synthetic oligonucleotide
gtaaatgggattccaacacgaacaa

SEQ ID NO: 18 - DNA, 24 bp, Artificial
  synthetic oligonucleotide
tgtcgtgcagtaagaacccaactc

(19) United States
(12) Patent Application Publication
(10) Pub. No.: US 2021/0009961 A1
(43) Pub. Date: Jan. 14, 2021

(54) Methods and Materials for Producing Hybrid Cell Lines

(71) Applicant: BioE LLC, Bloomington, MN (US)

(72) Inventors: Daniel Patrick Collins, Lino Lakes, MN (US); Clifford J. Steer, Eagan, MN (US)

(21) Appl. No.: 16/926,315

(22) Filed: Jul. 10, 2020

Related U.S. Application Data
(60) Provisional application No. 62/873,663, filed on Jul. 12, 2019.

(51) Int. Cl. C12N 5/16; G01N 33/50; C07K 14/76; C12P 13/02; G01N 33/68; G01N 33/62; C12M 1/24; C12M 1/00; A01N 1/02

(52) U.S. Cl. CPC C12N 5/16; C12N 2533/54; C07K 14/76; C12P 13/02; G01N 33/6803; G01N 33/62; C12M 23/08; C12M 23/14; A01N 1/0221; C12N 2506/14; C12N 2506/00; C12N 2501/39; C12N 2501/33; C12N 2500/24; C12N 2500/30; C12N 2501/11; C12N 2501/119; C12N 2501/12; C12N 2501/125; C12N 2501/237; C12N 2501/155; C12N 2501/2301; G01N 2333/76; G01N 33/5005

(57) ABSTRACT

Methods and materials are described for producing immortalized hybrid cells composed of a fusion of immortalized multi-lineage progenitor cells (MLPC) and primary hepatocytes. The hybrid cells express the biological activity of the primary hepatocytes and the immortality and expansion capacities of the immortalized MLPC. The methods of culture and expansion of the resultant hybrid cells and the methods to confirm the characteristics of the hybrid cells are described.

### CROSS-REFERENCE TO RELATED APPLICATIONS

This application claims the benefit of U.S. Provisional Application Ser. No. 62/873,663, filed Jul. 12, 2019. The disclosure of the prior application is considered part of (and is incorporated by reference in) the disclosure of this application.

### TECHNICAL FIELD

This document relates to methods and materials used to fuse immortalized cord blood-derived multi-lineage progenitor cells (MLPC) to somatic cells such as hepatocytes to create fused cell lines with the ability to grow and expand indefinitely while maintaining the biological characteristics of the somatic cells (e.g., hepatocyte characteristics).

### BACKGROUND

Livers and liver cells play an irreplaceable role in life-saving transplantation procedures and in drug discovery and toxicology. While there is a large need for donor livers to transplant, the actual availability (<8,000/yr) leaves that large need unmet. Liver cells, isolated from post-mortum donations, are used in drug discovery and toxicology due to the liver's central role in drug metabolism. Liver cells obtained for this purpose comprise a >$1 B industry supplying cells for drug developers and contract research organizations sub-contracting to larger drug developers. The inherent variability of the different lots of cells (liver cells are obtained from different donors) with regards to drug interactions complicate the interpretation of in vitro toxicology experiments. A different source for the creation of hepatocytes for both purposes (therapeutic and toxicologic) that is stable and reproducible could provide the platform needed for the development of them.

In the pursuit of the goal of a stable source of hepatocyte-like cells for drug discovery and toxicology studies, a number of different approaches have been taken with different sources of stem cells. The vast majority of commercial efforts have been utilizing either embryonic stem cells (ESC) or induced pluripotent stem cells (iPS). Other studies have suggested that cell sources including umbilical cord blood non-hematopoietic stem cells could provide a potential source of cells for the development of liver and pancreatic cells. Most of these efforts have resulted in cells that have significantly reduced function when compared to donor-derived primary hepatocytes or islet cells.

Currently, drug discovery and toxicology studies are dependent upon a renewable source of donated or cadaveric hepatocytes. They are critical to the industry, but they suffer from several insurmountable features. One limitation is that they are from individuals with different genetics and drug metabolism characteristics, and therefore are highly variable, and the resultant hepatocytes are a limited lot size that cannot be renewed. Hepatocytes have a limited life in vitro and do not divide and expand under current known culture conditions. Consistency, standardization and economy are great drivers for the development of these cells from genetically consistent sources like stem cells.

The process of differentiating stem cells to a specific cell type mostly results in a mixture of undifferentiated stem cells, stem cells that are not the desired phenotype, and the desired cells. It is important to separate these different cell types in instances of cellular therapy so as to prevent formation of teratomas (via undifferentiated stem cells), or atopic growth of cells in organs they shouldn't normally reside in. Additionally, stability of differentiated cells in their differentiated state has not been fully confirmed. Once differentiated, the cells need to be expanded many fold to produce a viable product and they must maintain their proper biological activity after the expansion. While undifferentiated ESC and iPS cells can be greatly expanded in their native state, after differentiation, expansion capacities are greatly reduced requiring serial differentiation of ESC or iPS cells and purification of hepatocyte-like cells in order to produce sufficient quantities of hepatocyte-like cells suitable for commercial applications.

### SUMMARY

This document is based, at least in part, on the discovery that human cord blood-derived multi-lineage progenitor cells (MLPC) can be immortalized using the nucleic acid encoding the telomerase reverse transcriptase (TERT) without inducing tumorigenesis or formation of teratomas, and while retaining their high proliferative potential and differentiation capacity of the wild-type cells. This document also is based, at least in part, on the discovery that fusing an immortalized MLPC (e.g., TERT-MLPC) to a somatic cell such as a primary hepatocyte or primary pancreatic cell results in an immortalized MLPC-hepatocyte fusion cell (also referred to as a hybrid cell herein) that combines the immortality and proliferative capacity of the TERT-MLPC while maintaining the biological activities and phenotype of the normal somatic cell. These hybrid cells do not require further differentiation and can be expanded indefinitely while maintaining their hepatocyte biological characteristics using a composition described herein (e.g., culture medium for the maintenance and expansion of such cells), facilitating large-scale expansion of these cells.

In one aspect, this document features a cell population that includes a plurality of hybrid cells, wherein each hybrid cell is composed of an immortalized MLPC and a primary somatic cell (e.g., primary hepatocyte). The hybrid cell can be created by the fusion of the immortalized MLPC and the primary somatic cell (e.g., hepatocyte). The immortalized MLPC can include a nucleic acid encoding a telomerase reverse transcriptase (e.g., a human telomerase reverse transcriptase). The hybrid cells can have the biological activity associated with the primary somatic cell. The hybrid cells can be immortalized and have the expandability of the immortalized MLPC. The hybrid cells can be expanded continuously in an expansion medium (e.g., an expansion medium that includes hydrocortisone, bovine serum albumin, insulin, transferrin, selenium, epithelial growth factor, basic fibroblast growth factor, fibroblast growth factor 4, hepatocyte growth factor, stem cell factor, oncostatin M, bone morphogenic protein 4, and interleukin 1 beta). The expansion medium further can include an antibiotic. The fusion cells can be positive for one or more (e.g., two, three, four, five, six, seven, eight, nine, ten, eleven, twelve, thirteen, fourteen, or all) of alkaline phosphatase, alpha fetoprotein, albumin, c-reactive protein, hepatocyte growth factor receptor, complement factor VII, complement factor IX, nestin, SOX-17, P450 CYP 1A2, P450 CYP 3A4, asialo-glycoprotein receptor 1, hepatocyte nuclear factor-4, GATA-4, and alpha-1-antitrypsin. The cell population can produce albumin and/or urea. The cell population can include a cryopreservative (e.g., fetal bovine serum, human serum, or human serum albumin in combination with one or more of the following: DMSO, trehalose, and dextran).

In another aspect, this document features a method of producing hybrid cells. The method includes a) combining immortalized multi-lineage progenitor cells and primary somatic cells (e.g., primary hepatocytes) in the presence of polyethylene glycol, b) culturing the mixture from step a) on a collagen-coated substrate; and c) selecting the hybrid cells that adhere to the substrate. The method further can include testing the hybrid cells for one or more characteristics of the primary somatic cells (e.g., primary hepatocytes). The hybrid cells can be tested for one or more of (e.g., two, three, four, five, six, seven, eight, nine, ten, eleven, twelve, thirteen, fourteen, or all) of alkaline phosphatase, alpha fetoprotein, albumin, c-reactive protein, hepatocyte growth factor receptor, complement factor VII, complement factor IX, nestin, SOX-17, P450 CYP 1A2, P450 CYP 3A4, asialo-glycoprotein receptor 1, hepatocyte nuclear factor-4, GATA-4, and alpha-1-antitrypsin. For example, the hybrid cells can be tested for albumin production and/or urea production.

This document also features a method of producing an expanded population of hybrid cells. The method includes providing a collagen-coated culturing device housing a purified population of hybrid cells, wherein each hybrid cell is composed of an immortalized MLPC and a primary hepatocyte, and culturing the hybrid cell in an expansion medium. The expansion medium can include hydrocortisone, bovine serum albumin, insulin, transferrin, selenium, epithelial growth factor, basic fibroblast growth factor, fibroblast growth factor 4, hepatocyte growth factor, stem cell factor, oncostatin M, bone morphogenic protein 4, and interleukin 1 beta. The hybrid cells can be tested for one or more of (e.g., two, three, four, five, six, seven, eight, nine, ten, eleven, twelve, thirteen, fourteen, or all) of alkaline phosphatase, alpha fetoprotein, albumin, c-reactive protein, hepatocyte growth factor receptor, complement factor VII, complement factor IX, nestin, SOX-17, P450 CYP 1A2, P450 CYP 3A4, asialo-glycoprotein receptor 1, hepatocyte nuclear factor-4, GATA-4, and alpha-1-antitrypsin. For example, the hybrid cells can be tested for albumin production and/or urea production.

This document also features articles of manufacture that include any of the cell populations described herein. The cell population can be housed within a container (e.g., a vial, bottle, or a bag) and further can include a cryopreservative (e.g., fetal bovine serum, human serum, or human serum albumin in combination with one or more of the following: DMSO, trehalose, and dextran).

This document also provides methods for immortalizing MLPC by inserting a nucleic acid encoding a telomerase reverse transcriptase (e.g., human TERT) and developing monotypic clonal cell lines from individual cells. Such immortalized cells (TERT-MLPC) can be fused with primary hepatocytes and long-lived cell lines can be established from the fused cells. This document also provides methods and materials for expanding these cell lines. This document also provides methods for cryopreserving the TERT-MLPC/hepatocyte fusion cells.

In one aspect, this document features a composition that includes a purified population of TERT-immortalized human fetal blood MLPC fused to a normal primary hepatocyte and a medium effective to facilitate expansion of the fused cell while retaining biological activity and phenotype consistent with normal hepatocytes. This embodiment features fusion of two cell types, wherein the undifferentiated TERT-MLPC are positive for CD9, CD105, CD106, CD90, negative for CD45, negative for CD34, and negative for SSEA-4 and the hepatocytes are positive, for example, for albumin, hepatocyte growth factor receptor, asialo glycoprotein receptor 1, hepatocyte nuclear factor 4, alpha-1-antitrypsin, complement factors 7 and 9. The expansion medium can include hydrocortisone, transferrin, insulin, selenium, epidermal growth factor, hepatocyte growth factor, stem cell factor, basic fibroblast growth factor, fibroblast growth factor-4, oncostatin M, bone morphogenic protein 4, and Interleukin 1 beta. The expansion medium further can include an antibiotic.

This document also features a method of expanding the hybrid cells that includes providing a collagen-coated culturing device housing a purified population of the fused cells and culturing the fused population of TERT-MLPC/hepatocytes with an expansion medium; a method of harvesting said cells from the culture vessel, and a method for cryopreservation of the cells.

In another aspect, this document includes a composition of an expanded population of fused cells. Such compositions further can include a cryopreservative (e.g., dimethylsulfoxide (DMSO) such as 1 to 10% DMSO). The cryopreservative can be fetal bovine serum, human serum, or human serum albumin in combination with one or more of the following: DMSO, trehalose, and dextran. For example, the cryopreservative can be human serum, DMSO, and trehalose, or fetal bovine serum and DMSO.

This document also features an article of manufacture that includes a clonal population of immortalized TERT-MLPC/hepatocyte fusion cells having a hepatocyte phenotype. The clonal population can be housed within a container (e.g., a vial or a bag). The container further can include a cryopreservative.

This embodiment also features a method of using the TERT-MLPC/hepatocyte fusion cells for commercial level cell production.

This embodiment also features a kit containing a basal medium (e.g., Williams E medium), growth factors (e.g., hydrocortisone, bovine serum albumin, insulin, transferrin, selenium, epithelial growth factor, basic fibroblast growth factor, fibroblast growth factor 4, hepatocyte growth factor, stem cell factor, oncostatin M, bone morphogenic protein 4 and interleukin 1 beta), and one or more antibiotics that can be combined to create a final expansion medium, as well as, instructions for use that would allow users of this medium to successfully expand their hepatocyte-like fusion cells.

Unless otherwise defined, all technical and scientific terms used herein have the same meaning as commonly understood by one of ordinary skill in the art to which this invention belongs. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DESCRIPTION OF DRAWINGS

FIG. 1 is a schematic of the procedural steps to isolate MLPC.

FIG. 2 contains photomicrographs of TERT-MLPC, hepatocytes and the TERT-MLPC/hepatocyte hybrid cells.

FIGS. 3A and 3B are photomicrographs of PCR products resolved on a 1% agarose gel and visualized under UV light.

### DETAILED DESCRIPTION

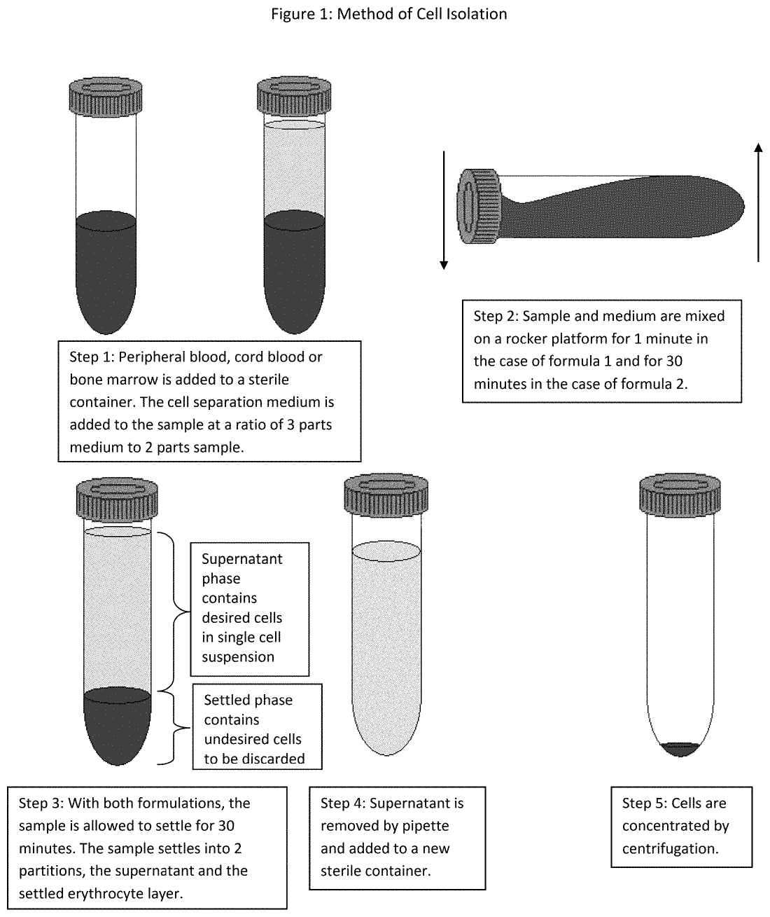

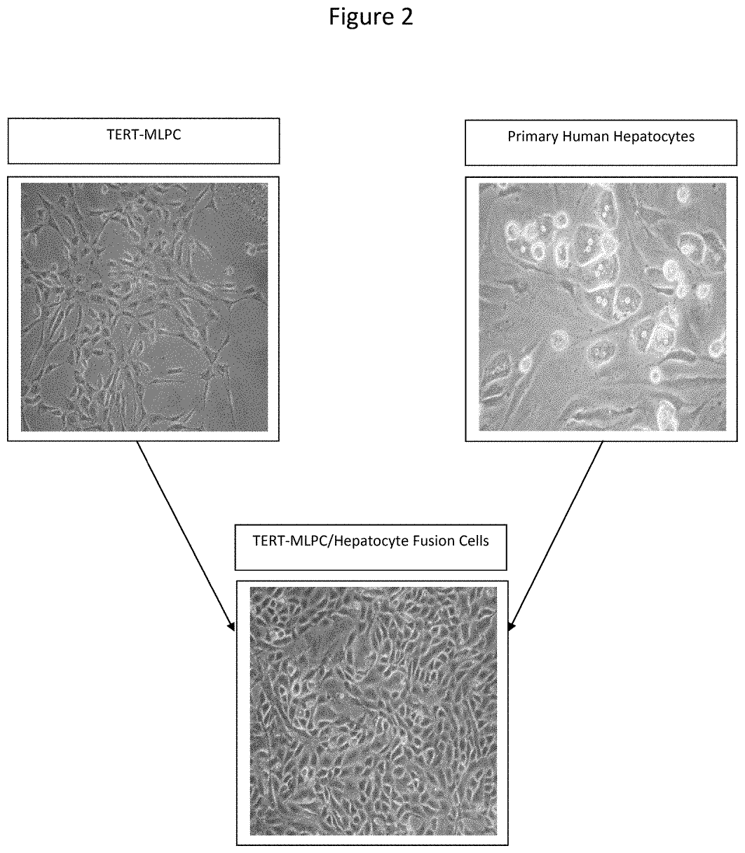

Multi-lineage progenitor cells (MLPC) are mesenchymal-like cells isolated from human umbilical cord blood. MLPC can be expanded more extensively than other sources of mesenchymal-like stem cells and have a much greater capacity for differentiation in comparison to other cell lineages outside of the well-described mesodermal outcomes. As described in U.S. Pat. Nos. 7,670,596, 7,622,108, and 7,875,54, fetal blood MLPC are distinguished from bone marrow-derived MSC, HSC, and USSC on the basis of their immunophenotypic characteristics, gene expression profile, morphology, and distinct growth pattern. MLPC can be isolated from fetal blood (e.g., cord blood) using the negative selection process and cell separation compositions disclosed in U.S. Pat. Nos. 7,160,723 and 7,476,547. See, for example, FIG. 1.

MLPC have a great expansive capacity, but are limited to 80-100 population doublings. This proliferative capacity is significantly different from other mesenchymal-like cells, including other cells from umbilical cord blood that are only capable of 20 population doublings. This capacity is enough to produce fairly large numbers of clonal (derived from a single cell) cell lines but not an unlimited number. MLPC have been shown to not be tumorigenic or induce teratomas in their native state.

As described herein, it was discovered that introducing the nucleic acid encoding telomerase reverse transcriptase (TERT) was able to immortalize the MLPC while retaining their high reproductive capacity (actually enhanced doubling time, with doubling time reduced from ˜36 hours to 22 hours), their non-tumorigenic properties, and extensive differentiation capacity; all properties of the original unmodified cell.

As described herein, techniques originally developed for producing monoclonal antibodies (typically a normal spleen-derived B-cell producing a specific antibody fused to a neoplastic myeloma cell to produce monoclonal antibodies) can be used to facilitate the direct fusion of the immortalized TERT-MLPC to normal primary somatic cells such as hepatocytes or pancreatic cells (e.g., using polyethylene glycol or electrofusion). See, for example, Pontecorvo, Somat Cell Mol Genet. 1975; 1:397-400; or Lu, Oncotarget, 2015 6(36): 38764-38776. Using such techniques and immortalized MLPC, fusion cells were produced that do not require further differentiation to the hepatocyte stage, can be expanded extensively and retain the biological characteristics of hepatocytes. The TERT-MLPC contributed the immortality and extensive expansion capacities while the hepatocytes contributed the biological functions associated with normal primary hepatocytes. Fusion cells retained 100% expression of hTERT as well as expression of mRNA, urea and albumin production, and intracellular marker expression characteristic of primary hepatocytes. These cells have been serially cultured for an extensive period without loss of hepatocyte biological activity nor their immortality.

The liver and pancreas develop from an embryonic endodermal tissue in the mid-thoracic area. The tissue that differentiates into the pancreas is most influenced by their proximity to the developing gastro-intestinal system. The tissue that develops into the liver is influenced by its proximity to the developing cardiac tissue. The systems that were developed to direct differentiation to these different tissue types were based on the development in the embryo. First, develop the undifferentiated cells into the common endodermal tissue of the two organs then add growth factors that would most closely replicate the conditions that cause the cells to divert their development pathway to either more fully developed liver or pancreatic tissues. In the case of ESC and iPS cells, differentiation to an end-point cell, like a hepatocyte, renders the cell to be limited in their replicative capacity after differentiation. Additionally, cells that have hepatocyte-like functions need to be isolated from the cells that either did not differentiate, or differentiated down a different cell type pathway. Success in mastering those techniques still requires repeated complicated culture conditions to replicate the final desired hepatocytes.

The discoveries described in this document by-pass the complicated culture conditions and produce a hybrid cell that has the biological characteristics of a normal hepatocyte, but with the added ability to be an immortalized cell line that can be banked as frozen stocks that need no further manipulation, and can be expanded to large numbers, sufficient for drug discovery and toxicology testing, or in a therapeutic role as part of an extra-corporeal cellular dialysis system.

The immortality and proliferative capacities are provided by the genome of the TERT-MLPC cells and the hepatocyte functional capacities are provided by the genome of the primary hepatocytes. In normal cell cultures, primary hepatocytes do not replicate and do not survive longer than 7-9 days. User instructions accompanying commercially available primary hepatocytes suggest that cells be used for CYP analysis should be investigated immediately after plating and completed in 3 days. Primary hepatocytes have reduced responsiveness after 5 days, and exhibit only minimal expansion capacities under current known culture conditions. MLPC/Hepatocyte fusion cells described herein have been frozen and recultured or continuously cultured for 9 months. In comparison to normal primary hepatocytes or hepatocyte-like cells developed from ESC or iPS cells, this is a highly unexpected outcome. It was not expected that a single clone of TERT-MLPC could provide both the proliferative capacity and the retention of differentiation capacity of the original MLPC and be capable of cell fusion and sharing of desired genetic traits with the fusion partner; in this case, hepatocytes. The fusion of TERT-MLPC to the hepatocytes working was highly unexpected because in the case of making monoclonal antibodies, the fusion is done between similar cells by lineage, one of them a neoplastic (cancer) cell.

The expansion medium described herein supports the expansion of these differentiated cells while maintaining the hepatocyte morphology, protein expression and preventing the reversion of the differentiated cells to more primitive states. The medium includes hydrocortisone, bovine serum albumin, insulin, transferrin, selenium, epithelial growth factor, basic fibroblast growth factor, fibroblast growth factor 4, hepatocyte growth factor, stem cell factor, oncostatin M, bone morphogenic protein 4 and interleukin 1 beta. For example, the medium can include about 5 mM hydrocortisone (e.g., hydrocortisone-21-hemisuccinate), about 80 ng/ml epithelial growth factor, about 20 ng/ml fibroblast growth factor basic, about 20 ng/ml fibroblast growth factor 4, about 40 ng/ml hepatocyte growth factor, about 40 ng/ml stem cell factor, about 20 ng/ml Oncostatin M, about 20 ng/ml bone morphogenic protein 4, and about 10 ng/ml Interleukin 1 beta. In some embodiments, the medium also can include an antibiotic and/or DMSO (e.g., 0.5%) and/or retinoic acid (e.g., 30 μg/ml).

### Cell Separation Compositions

MLPC can be isolated from fetal blood (e.g., cord blood) using the negative selection process and cell separation compositions disclosed in U.S. Pat. Nos. 7,160,723 and 7,476,547. Such cell compositions can include dextran and one or more antibodies against (i.e., that have binding affinity for) a cell surface antigen. Dextran is a polysaccharide consisting of glucose units linked predominantly in alpha (1 to 6) mode. Dextran can cause stacking of erythrocytes (i.e., rouleau formation) and thereby facilitate the removal of erythroid cells from solution. Antibodies against cell surface antigens can facilitate the removal of blood cells from solution via homotypic agglutination (i.e., agglutination of cells of the same cell type) and/or heterotypic agglutination (i.e., agglutination of cells of different cell types). For example, a cell separation composition can include dextran and antibodies against glycophorin A, CD15, and CD9. Cell separation compositions also can contain antibodies against other blood cell surface antigens including, for example, CD2, CD3, CD4, CD8, CD72, CD16, CD41a, HLA Class I, HLA-DR, CD29, CD11a, CD11b, CD11c, CD19, CD20, CD23, CD39, CD40, CD43, CD44, CDw49d, CD53, CD54, CD62L, CD63, CD66, CD67, CD81, CD82, CD99, CD100, Leu-13, TPA-1, surface Ig, and combinations thereof. Thus, cell separation compositions can be formulated to selectively agglutinate particular types of blood cells.

Typically, the concentration of anti-glycophorin A antibodies in a cell separation composition ranges from 0.1 to 15 mg/L (e.g., 0.1 to 10 mg/L, 1 to 5 mg/L, or 1 mg/L). Anti-glycophorin A antibodies can facilitate the removal of red cells from solution by at least two mechanisms. First, anti-glycophorin A antibodies can cause homotypic agglutination of erythrocytes since glycophorin A is the major surface glycoprotein on erythrocytes. In addition, anti-glycophorin A antibodies also can stabilize dextran-mediated rouleau formation. Exemplary monoclonal anti-glycophorin A antibodies include, without limitation, 107FMN (Murine IgG1 isotype), YTH89.1 (Rat IgG2b isotype), 2.2.2.E7 (Murine IgM isotype; BioE, St. Paul, Minn.), and E4 (Murine IgM isotype). See e.g., M. Vanderlaan et al., Molecular Immunology 20:1353 (1983); Telen M. J. and Bolk, T. A., Transfusion 27: 309 (1987); and Outram S. et al., Leukocyte Research. 12:651 (1988).

The concentration of anti-CD15 antibodies in a cell separation composition can range from 0.1 to 15 mg/L (e.g., 0.1 to 10, 1 to 5, or 1 mg/L). Anti-CD15 antibodies can cause homotypic agglutination of granulocytes by crosslinking CD15 molecules that are present on the surface of granulocytes. Anti-CD15 antibodies also can cause homotypic and heterotypic agglutination of granulocytes with monocytes, NK-cells and B-cells by stimulating expression of adhesion molecules (e.g., L-selectin and beta-2 integrin) on the surface of granulocytes that interact with adhesion molecules on monocytes, NK-cells and B-cells. Heterotypic agglutination of these cell types can facilitate the removal of these cells from solution along with red cell components. Exemplary monoclonal anti-CD15 antibodies include, without limitation, AHN1.1 (Murine IgM isotype), FMC-10 (Murine IgM isotype), BU-28 (Murine IgM isotype), MEM-157 (Murine IgM isotype), MEM-158 (Murine IgM isotype), 324.3.B9 (Murine IgM isotype; BioE, St. Paul, Minn.), and MEM-167 (Murine IgM isotype). See e.g., Leukocyte typing IV (1989); Leukocyte typing II (1984); Leukocyte typing VI (1995); Solter D. et al., Proc. Natl. Acad. Sci. USA 75:5565 (1978); Kannagi R. et al., J. Biol. Chem. 257:14865 (1982); Magnani, J. L. et al., Arch. Biochem. Biophys 233:501 (1984); Eggens I. et al., J. Biol. Chem. 264:9476 (1989).

The concentration of anti-CD9 antibodies in a cell separation composition can range from 0.1 to 15, 0.1 to 10, 1 to 5, or 1 mg/L. Anti-CD9 antibodies can cause homotypic agglutination of platelets. Anti-CD9 antibodies also can cause heterotypic agglutination of granulocytes and monocytes via platelets that have adhered to the surface of granulocytes and monocytes. CD9 antibodies can promote the expression of platelet p-selectin (CD62P), CD41/61, CD31, and CD36, which facilitates the binding of platelets to leukocyte cell surfaces. Thus, anti-CD9 antibodies can promote multiple cell-cell linkages and thereby facilitate agglutination and removal from solution. Exemplary monoclonal anti-CD9 antibodies include, without limitation, MEM-61 (Murine IgG1 isotype), MEM-62 (Murine IgG1 isotype), MEM-192 (Murine IgM isotype), FMC-8 (Murine IgG2a isotype), SN4 (Murine IgG1 isotype), 8.10.E7 (Murine IgM isotype; BioE, St. Paul, Minn.), and BU-16 (Murine IgG2a isotype). See e.g., Leukocyte typing VI (1995); Leukocyte typing II (1984); Von dem Bourne A. E. G. Kr. and Moderman P. N. (1989) In Leukocyte typing IV (ed. W. Knapp, et al), pp. 989-92, Oxford University Press, Oxford; Jennings, L. K., et al. In Leukocyte typing V, ed. S. F. Schlossmann et al., pp. 1249-51, Oxford University Press, Oxford (1995); Lanza F. et al., J. Biol. Chem. 266:10638 (1991); Wright et al., Immunology Today 15:588 (1994); Rubinstein E. et al., Seminars in Thrombosis and Hemostasis 21:10 (1995).

In some embodiments, a cell separation composition contains antibodies against CD41, which can selectively agglutinate platelets. In some embodiments, a cell separation composition contains antibodies against CD3, which can selectively agglutinate T-cells. In some embodiments, a cell separation composition contains antibodies against CD2, which can selectively agglutinate T-cells and NK cells. In some embodiments, a cell separation composition contains antibodies against CD72, which can selectively agglutinate B-cells. In some embodiments, a cell separation composition contains antibodies against CD16, which can selectively agglutinate NK cells and neutrophilic granulocytes. The concentration of each of these antibodies can range from 0.01 to 15 mg/L. Exemplary anti-CD41 antibodies include, without limitation, PLT-1 (Murine IgM isotype), CN19 (Murine IgG₁ isotype), and 8.7.C3 (Murine IgG1 isotype). Non-limiting examples of anti-CD3 antibodies include OKT3 (Murine IgG₁), HIT3a (Murine IgG2a isotype), SK7 (Murine IgG₁) and BC3 (Murine IgG₂ₐ). Non-limiting examples of anti-CD2 antibodies include 7A9 (Murine IgM isotype), T11 (Murine IgG₁ isotype), and Leu5b (Murine IgG₂ₐ Isotype). Non-limiting examples of anti-CD72 antibodies include BU-40 (Murine IgG₁ isotype) and BU-41 (Murine IgG₁ isotype). Non-limiting examples of anti-CD16 antibodies include 3G8 (Murine IgG).

As mentioned above, cell separation compositions can be formulated to selectively agglutinate particular blood cells. As an example, a cell separation composition containing antibodies against glycophorin A, CD15, and CD9 can facilitate the agglutination of erythrocytes, granulocytes, NK cells, B cells, and platelets. T cells, NK cells, and rare precursor cells such as MLPC, then can be recovered from solution. If the formulation also contained an antibody against CD3, T cells also could be agglutinated, and NK cells and rare precursors such as MLPC could be recovered from solution.

Cell separation compositions can contain antibodies against surface antigens of other types of cells (e.g., cell surface proteins of tumor cells). Those of skill in the art can use routine methods to prepare antibodies against cell surface antigens of blood, and other, cells from humans and other mammals, including, for example, non-human primates, rodents (e.g., mice, rats, hamsters, rabbits and guinea pigs), swine, bovines, and equines.

Typically, antibodies used in the composition are monoclonal antibodies, which are homogeneous populations of antibodies to a particular epitope contained within an antigen. Suitable monoclonal antibodies are commercially available, or can be prepared using standard hybridoma technology. In particular, monoclonal antibodies can be obtained by techniques that provide for the production of antibody molecules by continuous cell lines in culture, including the technique described by Kohler, G. et al., Nature, 1975, 256:495, the human B-cell hybridoma technique (Kosbor et al., Immunology Today 4:72 (1983); Cole et al., Proc. Natl. Acad. Sci. USA 80:2026 (1983)), and the EBV-hybridoma technique (Cole et al., “Monoclonal Antibodies and Cancer Therapy,” Alan R. Liss, Inc., pp. 77-96 (1983)).

Antibodies can be of any immunoglobulin class including IgG, IgM, IgE, IgA, IgD, and any subclass thereof. Antibodies of the IgG and IgM isotypes are particularly useful in cell separation compositions of the invention. Pentameric IgM antibodies contain more antigen binding sites than IgG antibodies and can, in some cases (e.g., anti-glycophorin A and anti-CD15), be particularly useful for cell separation reagents. In other cases (e.g., anti-CD9 antibodies), antibodies of the IgG isotype are particularly useful for stimulating homotypic and/or heterotypic agglutination.

Antibodies against cell surface antigens can be provided in liquid phase (i.e., soluble). Liquid phase antibodies typically are provided in a cell separation composition at a concentration between about 0.1 and about 15 mg/l (e.g., between 0.25 to 10, 0.25 to 1, 0.5 to 2, 1 to 2, 4 to 8, 5 to 10 mg/l).

Antibodies against cell surface antigens also can be provided in association with a solid phase (i.e., substrate-bound). Antibodies against different cell surface antigens can be covalently linked to a solid phase to promote crosslinking of cell surface molecules and activation of cell surface adhesion molecules. The use of substrate-bound antibodies can facilitate cell separation (e.g., by virtue of the mass that the particles contribute to agglutinated cells, or by virtue of properties useful for purification).

In some embodiments, the solid phase with which a substrate-bound antibody is associated is particulate. In some embodiments, an antibody is bound to a latex microparticle (0.5 to 10 microns in diameter) such as a paramagnetic bead (e.g., via biotin-avidin linkage, covalent linkage to COO groups on polystyrene beads, or covalent linkage to NH₂ groups on modified beads). In some embodiments, an antibody is bound to an acid-etched glass particle (e.g., via biotin-avidin linkage). In some embodiments, an antibody is bound to an aggregated polypeptide such as aggregated bovine serum albumin (e.g., via biotin-avidin linkage, or covalent linkage to polypeptide COO groups or NH₂ groups). In some embodiments, an antibody is covalently linked to a polysaccharide such as high molecular weight (e.g., >1,000,000 Mᵣ) dextran sulfate. In some embodiments, biotinylated antibodies are linked to avidin particles, creating tetrameric complexes having four antibody molecules per avidin molecule. In some embodiments, antibodies are bound to biotinylated agarose gel particles (One Cell Systems, Cambridge, Mass., U.S.A.) via biotin-avidin-biotinylated antibody linkages. Such particles typically are about 300-500 microns in size, and can be created in a sonicating water bath or in a rapidly mixed water bath.

Cell-substrate particles (i.e., particles including cells and substrate-bound antibodies) can sediment from solution as an agglutinate. Cell-substrate particles also can be removed from solution by, for example, an applied magnetic field, as when the particle is a paramagnetic bead. Substrate-bound antibodies typically are provided in a cell separation composition at a concentration between about 0.1 and about 50.0×10⁹ particles/l (e.g., between 0.25 to 10.0×10⁹, 1 to 20.0×10⁹, 2 to 10.0×10⁹, 0.5 to 2×10⁹, 2 to 5×10⁹, 5 to 10×10⁹, and 10 to 30×10⁹ particles/l), where particles refers to solid phase particles having antibodies bound thereto thereto by previously described methods.

Cell separation compositions also can contain divalent cations (e.g., Ca⁺² and Mg⁺²). Divalent cations can be provided, for example, by a balanced salt solution (e.g., Hank's balanced salt solution). Ca⁺² ions reportedly are important for selectin-mediated and integrin-mediated cell-cell adherence.

Cell separation compositions also can contain an anticoagulant such as heparin. Heparin can prevent clotting and non-specific cell loss associated with clotting in a high calcium environment. Heparin also promotes platelet clumping. Clumped platelets can adhere to granulocytes and monocytes and thereby enhance heterotypic agglutination more so than single platelets. Heparin can be supplied as a heparin salt (e.g., sodium heparin, lithium heparin, or potassium heparin).

### Populations and Clonal Lines of MLPC

MLPC can be purified from human fetal blood using a cell separation composition described above. As used herein, “purified” means that at least 90% (e.g., 91, 92, 93, 94, 95, 96, 97, 98, or 99%) of the cells within the population are MLPC. As used herein, “MLPC” refers to fetal blood cells that are positive for CD9 and typically display a constellation of other markers such as CD13, CD73, and CD105. “MLPC population” refers to the primary culture obtained from the human fetal blood and uncloned progeny thereof. “Clonal line” refers to a cell line derived from a single cell. As used herein, a “cell line” is a population of cells able to renew themselves for extended periods of times in vitro under appropriate culture conditions. The term “line,” however, does not indicate that the cells can be propagated indefinitely. Rather, clonal lines described herein typically can undergo 75 to 100 doublings before senescing and without losing the properties of hepatocyte cells for this duration of propagation.

Typically, an MLPC population is obtained by contacting a fetal blood sample with a cell separation composition described above and allowing the sample to partition into an agglutinate and a supernatant phase. For example, the sample can be allowed to settle by gravity or by centrifugation. Preferably, MLPC are purified from an umbilical cord blood sample that is less than 48 hours old (e.g., less than 24, 12, 8, or 4 hours post-partum). After agglutination, unagglutinated cells can be recovered from the supernatant phase. For example, cells in the supernatant phase can be recovered by centrifugation then washed with a saline solution and plated on a solid substrate (e.g., a plastic culture device such as a chambered slide or culture flask), using a standard growth medium with 10% serum (e.g., DMEM with 10% serum; RPMI-1640 with 10% serum, or mesenchymal stem cell growth medium with 10% serum (catalog # PT-3001, Cambrex, Walkersville, Md.). MLPC attach to the surface of the solid substrate while other cells, including T cells, NK cells and CD34⁺ HSC, do not and can be removed with washing. The MLPC change from the leukocyte morphology to the fibroblastic morphology between 3 days and 2 weeks post initiation of culture after which the cells enter logarithmic growth phase and will continue growing logarithmically as long as cultures are maintained at cell concentrations of less than about 1.5×10⁵ cells/cm².

Clonal lines can be established by harvesting the MLPC then diluting and re-plating the cells on a multi-well culture plate such that a single cell can be found in a well. Cells can be transferred to a larger culture flask after a concentration of 1 to 5×10⁵ cells/75 cm² is reached. Cells can be maintained at a concentration between 1×10⁵ and 5×10⁵ cells/75 cm² for logarithmic growth. See, e.g., U.S. Pat. Nos. 7,670,596 and 7,632,108

MLPC, TERT-MLPC, or hybrid cells (e.g., a fusion between TERT-MLPC and primary hepatocytes) can be assessed for viability, proliferation potential, and longevity using techniques known in the art. For example, viability can be assessed using trypan blue exclusion assays, fluorescein diacetate uptake assays, or propidium iodide uptake assays. Proliferation can be assessed using thymidine uptake assays or MTT cell proliferation assays. Longevity can be assessed by determining the maximum number of population doublings of an extended culture.

MLPC, TERT-MLPC, or hybrid cells (e.g., a fusion between TERT-MLPC and primary hepatocytes) can be immunophenotypically characterized using known techniques. For example, the cell culture medium can be removed from the tissue culture device and the adherent cells washed with a balanced salt solution (e.g., Hank's balanced salt solution) and bovine serum albumin (e.g., 2% BSA). Cells can be incubated with an antibody having binding affinity for a cell surface antigen such as hepatocyte growth factor receptor or asialo-glycoprotein receptor 1, or any other cell surface antigen. In some embodiments, cells can be fixed and permeabilized and incubated with an antibody that binds to an antigen internal to the cell. An antibody can be detectably labeled (e.g., fluorescently or enzymatically) or can be detected using a secondary antibody that is detectably labeled. Alternatively, the cell surface antigens on a cell population can be characterized using flow cytometry and fluorescently labeled antibodies.

As described herein, the cell surface antigens present on MLPC can vary, depending on the stage of culture. Early in culture when MLPC display a leukocyte-like morphology, MLPC are positive for CD9 and CD45, SSEA-4 (stage-specific embryonic antigen-4), CD34, as well as CD13, CD29, CD44, CD73, CD90, CD105, stem cell factor, STRO-1 (a cell surface antigen expressed by bone marrow stromal cells), SSEA-3 (galactosylgloboside), and CD133, and are negative for CD15, CD38, glycophorin A (CD235a), and lineage markers CD2, CD3, CD4, CD5, CD7, CD8, CD10, CD11b, CD16, CD19, CD20, CD21, CD22, CD33, CD36, CD41, CD61, CD62E, CD72, HLA-DR, and CD102. After transition to the fibroblastic morphology, MLPC remain positive for CD9, CD13, CD29, CD73, CD90, and CD105, are positive for CD106, and become negative for CD34, CD41, CD45, stem cell factor, STRO-1, SSEA-3, SSEA-4, and CD133. At all times during in vitro culture, the undifferentiated MLPC are negative for CD15, CD38, glycophorin A (CD235a), and lineage markers CD2, CD3, CD4, CD5, CD7, CD8, CD10, CD11b, CD16, CD19, CD20, CD21, CD22, CD33, CD36, CD41, CD61, CD62E, CD72, HLA-DR, and CD102.

Bone marrow-derived MSC and MAPC as well as the cord blood-derived USSC have been described as being derived from a CD45⁻/CD34⁻ cell population. MLPC are distinguished from those cell types as being a CD45⁺/CD34⁺ derived cell. Additionally, the presence and persistence of CD9 on the fetal blood-derived MLPC at all stages of maturation further distinguishes MLPC from MSC and MAPC, which do not possess CD9 as a marker. CD9 is expressed as a marker on human embryonic stem cells. MLPC, which share the hematopoietic markers CD45, CD133, CD90 and CD34 during their leukocyte morphology phase, can be distinguished from HSC by their obligate plastic adherence and the presence of mesenchymal associated markers CD105, CD29, CD73, CD13 and embryonic associated markers SSEA-3 and SSEA-4. Additionally using currently available technology, HSC are unable to be cultured in vitro without further differentiation while MLPC can be expanded for many generations without differentiation. MLPC also differ from MSC and USSC by their more gracile in vitro culture appearance, thread-like cytoplasmic projections and their preference for low density culture conditions for optimal growth.

MLPC, TERT-MLPC, or hybrid cells also can be characterized based on the expression of one or more genes. Methods for detecting gene expression can include, for example, measuring levels of the mRNA or protein of interest (e.g., by Northern blotting, reverse-transcriptase (RT)-PCR, microarray analysis, Western blotting, ELISA, or immunohistochemical staining). The gene expression profile of MLPC is significantly different than other cell types Microarray analysis indicated that the MLPC lines have an immature phenotype that differs from the phenotypes of, for example, CD133+ HSC, lineage negative cells (Forraz et al., Stem Cells, 22(1):100-108 (2004)), and MSC (catalog #PT-2501, Cambrex, Walkersville, Md., U.S. Pat. No. 5,486,359), which demonstrate a significant degree of commitment down several lineage pathways. See, e.g., U.S. Pat. Nos. 7,670,596 and 7,622,108

Comparison of the gene expression profile of MLPC and MSC demonstrates MSC are more committed to connective tissue pathways. There are 80 genes up-regulated in MSC, and 152 genes up-regulated in MLPC. In particular, the following genes were up-regulated in MLPC when compared with MSC, i.e., expression was decreased in MSC relative to MLPC: ITGB2, ARHGAP9, CXCR4, INTEGRINB7, PECAM1, PRKCB_1, PRKCB_3, IL7R, AIF1, CD45_EX10-11, PLCG2, CD37, PRKCB_2, TCF2_1, RNF138, EAAT4, EPHA1, RPLP0, PTTG, SERPINA1_2, ITGAX, CD24, F11R, RPL4, ICAM1, LMO2, HMGB2, CD38, RPL7A, BMP3, PTHR2, S100B, OSF, SNCA, GRIK1, HTR4, CHRM1, CDKN2D, HNRPA1, IL6R, MUSLAMR, ICAM2, CSK, ITGA6, MMP9, DNMT1, PAK1, IKKB, TFRC_MIDDLE, CHI3L2, ITGA4, FGF20, NBR2, TNFRSF1B, CEBPA_3, CDO1, NFKB1, GATA2, PDGFRB, ICSBP1, KCNE3, TNNC1, ITGA2B, CCT8, LEFTA, TH, RPS24, HTR1F, TREM1, CCNB2, SELL, CD34, HMGIY, COX7A2, SELE, TNNT2, SEM2, CHEK1, CLCN5, F5, PRKCQ, ITGAL, NCAM2, ZNF257-MGC12518-ZNF92-ZNF43-ZNF273-FLJ90430, CDK1, RPL6, RPL24, IGHA1-IGHA2_M, PUM2, GJA7, HTR7, PTHR1, MAPK14, MSI2_1, KCNJ3, CD133, SYP, TFRC_5PRIME, TDGF1-TDGF3_2, FLT3, HPRT, SEMA4D, ITGAM, KIAA0152_3, ZFP42, SOX20, FLJ21190, CPN2, POU2F2, CASP8_1, CLDN10, TREM2, TERT, OLIG1, EGR2, CD44_EX3-5, CD33, CNTFR, OPN, COL9A1_2, ROBO4, HTR1D_1, IKKA, KIT, NPPA, PRKCH, FGF4, CD68, NUMB, NRG3, SALL2, NOP5, HNF4G; FIBROMODULIN, CD58, CALB1, GJB5, GJA5, POU5F_1, GDF5, POU6F1, CD44_EX16-20, BCAN, PTEN1-PTEN2, AGRIN, ALB, KCNQ4, DPPA5, EPHB2, TGFBR2, and ITGA3. See, e.g., U.S. Pat. Nos. 7,670,596 and 7,622,108. CD106 are positive in MLPC and negative in MSC. Confocal analysis of stem-cell-associated markers SOX-2 and Oct 3/4 show distinctive differences between the MSC and MLPC. MSC are negative for both SOX-2 and Oct 3/4, while MLPC are positive for both.

MLPC express a number of genes associated with “stemness,” which refers to the ability to self-renew undifferentiated and ability to differentiate into a number of different cell types. Genes associated with “stemness” include the genes known to be over-expressed in human embryonic stem cells, including, for example, POU5F (Oct4), TERT, and ZFP42. For example, 65 genes associated with protein synthesis are down-regulated, 18 genes linked with phosphate metabolism are down-regulated, 123 genes regulating proliferation and cell cycling are down-regulated, 12 different gene clusters associated with differentiation surface markers are down-regulated, e.g., genes associated with connective tissue, including integrin alpha-F, laminin and collagen receptor, ASPIC, thrombospondins, endothelium endothelin-1 and -2 precursors, epidermal CRABP-2, and genes associated with adipocytes, including, for example, the leptin receptor, and 80 genes linked to nucleic acid binding and regulation of differentiation are up-regulated. Thus, the immaturity of a population of MLPC can be characterized based on the expression of one or more genes (e.g., one or more of CXCR4, FLT3, TERT, KIT, POU5F, or hematopoietic CD markers such as CD9, CD34, and CD133). See, e.g., U.S. Pat. Nos. 7,670,596 and 7,622,108

### Modified Populations of MLPC

MLPC can be modified such that the cells can produce one or more polypeptides or other therapeutic compounds of interest. To modify the isolated cells such that a polypeptide or other therapeutic compound of interest is produced, the appropriate exogenous nucleic acid must be delivered to the cells. In some embodiments, the cells are transiently transfected, which indicates that the exogenous nucleic acid is episomal (i.e., not integrated into the chromosomal DNA). In other embodiments, the cells are stably transfected, i.e., the exogenous nucleic acid is integrated into the host cell's chromosomal DNA The term “exogenous” as used herein with reference to a nucleic acid and a particular cell refers to any nucleic acid that does not originate from that particular cell as found in nature. In addition, the term “exogenous” includes a naturally occurring nucleic acid. For example, a nucleic acid encoding a polypeptide that is isolated from a human cell is an exogenous nucleic acid with respect to a second human cell once that nucleic acid is introduced into the second human cell. The exogenous nucleic acid that is delivered typically is part of a vector in which a regulatory element such as a promoter is operably linked to the nucleic acid of interest.

Cells can be engineered using a viral vector such as an adenovirus, adeno-associated virus (AAV), retrovirus, lentivirus, vaccinia virus, measles viruses, herpes viruses, or bovine papilloma virus vector. See, Kay et al. (1997) Proc. Natl. Acad. Sci. USA 94:12744-12746 for a review of viral and non-viral vectors. A vector also can be introduced using mechanical means such as liposomal or chemical mediated uptake of the DNA. For example, a vector can be introduced into an MLPC by methods known in the art, including, for example, transfection, transformation, transduction, electroporation, infection, microinjection, cell fusion, DEAE dextran, calcium phosphate precipitation, liposomes, LIPOFECTIN™, lysosome fusion, synthetic cationic lipids, use of a gene gun or a DNA vector transporter.

A vector can include a nucleic acid that encodes a selectable marker. Non-limiting examples of selectable markers include puromycin, adenosine deaminase (ADA), aminoglycoside phosphotransferase (neo, G418, APH), dihydrofolate reductase (DHFR), hygromycin-B-phosphtransferase, thymidine kinase (TK), and xanthin-guanine phosphoribosyltransferase (XGPRT). Such markers are useful for selecting stable transformants in culture.

MLPC also can have a targeted gene modification. Homologous recombination methods for introducing targeted gene modifications are known in the art. To create a homologous recombinant MLPC, a homologous recombination vector can be prepared in which a gene of interest is flanked at its 5′ and 3′ ends by gene sequences that are endogenous to the genome of the targeted cell, to allow for homologous recombination to occur between the gene of interest carried by the vector and the endogenous gene in the genome of the targeted cell. The additional flanking nucleic acid sequences are of sufficient length for successful homologous recombination with the endogenous gene in the genome of the targeted cell. Typically, several kilobases of flanking DNA (both at the 5′ and 3′ ends) are included in the vector. Methods for constructing homologous recombination vectors and homologous recombinant animals from recombinant stem cells are commonly known in the art (see, e.g., Thomas and Capecchi, 1987, Cell 51:503; Bradley, 1991, Curr. Opin. Bio/Technol. 2:823-29; and PCT Publication Nos. WO 90/11354, WO 91/01140, and WO 93/04169.

Bone marrow derived MSC and MSC-like cells have been shown to be susceptible to cellular senescence both in vitro and in vivo, with differential capacity decreasing with age. This senescence has been linked to telomere shortening and reduced telomerase activity. Telomerase adds a six-base DNA repeat sequence TTAGGG to the ends of chromosomes preserving their integrity. Telomerase consists of several proteins including a 120 kDa catalytic subunit known as telomerase reverse transcriptase (TERT). Telomerase activity has been shown to be increased in many tumors and has been suggested to play a key role in prevention of cellular senescence. It has been demonstrated that insertion of the gene for TERT can extend the proliferative potential of primary human cells, such as fibroblasts, endothelial cells and chondrocytes without causing neoplastic transformation. More recently retroviral vector-mediated hTERT expression in bone marrow derived MSC cells prolonged the life span of these cells without affecting differentiation both in vitro and in vivo. Additionally, it has been reported that transduction of mesenchymal stem cells with hTERT increased the osteogenic potential of mesenchymal stem cells with a coincidental increase in osteogenic related gene expression. In this embodiment, MLPC were transduced with hTERT in a bid to prolong life span without affecting differential capacity. Differentiated TERT-MLPC had similar or enhanced differentiation-associated gene expression compared to non-transduced MLPC and maintained TERT expression and telomere length after differentiation. The fusion of TERT-MLPC to the hepatocytes working was highly unexpected. The fact that fusion performed as expected was also surprising.

Results have been generated providing good to excellent production of albumin from the MLPC differentiated cells, even better production from the MLPC/Hepatocyte fusion cells, and also good results from the Marmoset cells. Results are shown proof positive for liver cells (they are the only cells that can make albumin).

TERT-MLPC or hybrid cells described herein can be cryopreserved by suspending the cells (e.g. 5×10⁶ to 2×10⁷ cells/mL) in a cryopreservative such as dimethylsulfoxide (DMSO, typically 1 to 10%) or in fetal bovine serum, human serum, or human serum albumin in combination with one or more of DMSO, trehalose, and dextran. For example, (1) fetal bovine serum containing 10% DMSO; (2) human serum containing 10% DMSO and 1% Dextran; (3) human serum containing 1% DMSO and 5% trehalose; or (4) 20% human serum albumin, 1% DMSO, and 5% trehalose can be used to cryopreserve TERT-MLPC or the hybrid cells. After adding cryopreservative, the cells can be frozen (e.g., to −90° C.). In some embodiments, the cells are frozen at a controlled rate (e.g., controlled electronically or by suspending the cells in a bath of 70% ethanol and placed in the vapor phase of a liquid nitrogen storage tank. When the cells are chilled to −90° C., they can be placed in the liquid phase of the liquid nitrogen storage tank for long term storage. Cryopreservation can allow for long-term storage of these cells for therapeutic use.

The invention will be further described in the following examples, which do not limit the scope of the invention described in the claims.

### EXAMPLES

### Example 1: Purification of MLPC

The cell separation reagent of Table 1 was used to isolate MLPC from the non-agglutinated supernatant phase.

TABLE 1
Cell Separation Reagent

| | | |
|---|---|---|
| Dextran (average molecular weight 413,000) | 20 | g/l |
| Hank's balanced salt solution (pH 7.2-7.4) | 50 | ml/l |

| | |
|---|---|
| Anti-CD15 | 0.1-15 mg/L (e.g., |
| (murine IgM monoclonal antibody, clone 324.3.B9) | about 2.0 mg/L) |

Briefly, 50-150 ml of CPDA anti-coagulated umbilical cord blood (<48 hours old) was gently mixed with an equal volume of cell separation composition described in Table 1 for 30 minutes. After mixing was complete, the container holding the blood/cell separation composition mixture was placed in an upright position and the contents allowed to settle by normal 1×g gravity for 30 minutes. After settling was complete, the non-agglutinated cells were collected from the supernatant. The cells were recovered from the supernatant by centrifugation then washed with PBS. Cells were resuspended in complete MSCGM (Mesenchymal stem cell growth medium, catalog # PT-3001, Cambrex, Walkersville, Md.) and adjusted to 2-9×10⁶ cells/ml with complete MSCGM. Cells were plated in a standard plastic tissue culture flask (e.g., Corning), chambered slide, or other culture device and allowed to incubate overnight at 37° C. in a 5% CO₂ humidified atmosphere. All subsequent incubations were performed at 37° C. in a 5% CO₂ humidified atmosphere unless otherwise noted. MLPC attached to the plastic during this initial incubation. Non-adherent cells (T-cells, NK-cells and CD34+ hematopoietic stem cells) were removed by vigorous washing of the flask or well with complete MSCGM.

MLPC cultures were fed periodically by removal of the complete MSCGM and addition of fresh complete MSCGM. Cells were maintained at concentrations of 1×10⁵-1×10⁶ cells/75 cm² by this method. When cell cultures reached a concentration of 8×10⁵ -1×10⁶ cells/75 cm², cells were cryopreserved using 10% DMSO and 90% serum or expanded into new flasks. Cells were recovered from the adherent cultures by removal of the complete MSCGM and replacement with Tryp-LE (Gibco). Cells were incubated for 15-60 minutes at 37° C. then collected from the flask and washed in complete MSCGM. Cells were then replated at 1×10⁵ cells/mL. Cultures that were allowed to achieve confluency were found to have diminished capacity for both proliferation and differentiation. Subsequent to this finding, cultures were not allowed to achieve higher densities than 5×10⁶ cells/75 cm².

### Example 2: Immortalization of MLPC by the Insertion of the TERT Gene

pRRLsin.hCMV hTERT lentiviral expression plasmid (Dr. Noriyuki Kasahara, Dept of Medicine, UCLA, CA) was used in this experiment. The telomerase vector was produced by three plasmid transient transductions using 10 μg of the self-inactivating (sin) hTERT lentiviral expression plasmid, 10 μg of the gag/pol plasmid, pCMV delta 8.2, and 2 μg of the envelope plasmid, pCMV VSVG, in a calcium phosphate transduction protocol according to the manufacturer's directions (Clontech). HEK 293T cells at 60-70% confluence in a 10 cm dish were given 10 ml fresh medium (DMEM, 10% FBS without antibiotics) 3-4 hours prior to transduction. After incubation of transduced cells overnight at 37° C., 5% CO₂, the medium was replaced with 6 ml fresh medium and incubated for an additional 24 hours. The supernatant was then collected and passed through a 0.45 μm filter and stored at −80° C. until used for transduction.

Mixed MLPC cultures (passage 12) were seeded at a density of 5×10⁴ cells/well of a 6-well tissue culture dish in complete MSCGM (without antibiotics) 24-hours prior to transduction. The vector supernatant was diluted 1:10 with DMEM 10% FBS containing 8 μg/ml polybrene and 1 ml was added to each well from which the growth medium had been removed. After 4 hours at 37° C., 5% CO₂, the diluted vector was removed and replaced with MSCGM. To get an estimate of transduction efficiency, MLPC were also transduced identically in parallel with pRRL sinhCMV GFP vector supernatants which had been serially diluted. GFP expression was analyzed 60 hours after transduction using FACS analysis. The titer of the GFP vector supernatant was 5×10⁴.

### Establishment of MLPC-TERT Cell Lines

As with the non-transfected MLPC, clonal MLPC-TERT cell lines were developed by limited dilution cloning. Wells with only one detectable cell were propagated in larger culture vessels to achieve cell numbers sufficient for analysis. Of the ten stable cell lines that were developed, one clone exhibited the combined characteristics of immortality and differentiation outside of mesodermal outcomes, E12-TERT (E12). This cell line has been in culture for multiple years and was used in some of the experiments described herein.

### Example 3: Fusion of TERT-MLPC With Primary Human Hepatocytes

Cryo-preserved primary human hepatocytes were obtained from Sekisui XenoTech (Kansas City, Kans.). Cells were thawed with OptiThaw medium and enumerated with OptiCount medium in a standard hemacytometer. Cells were diluted to a final concentration of 106 cells/ml of Opti-Plate medium. Cells were plated in collagen-coated 6 well plates (BD Biosciences) at 1 ml per well. After 4 hours of plating, the medium was changed to OptiCulture medium for the duration of culture. Medium was exchanged every 24 hours.

TERT-MLPC (2-5×10⁶) were mixed with an equal number of the primary human hepatocytes and pelleted at 300×g for 5 minutes. Cells were washed twice with 10 ml RPMI+0.01% EDTA. Cells were again pelleted and the supernatant removed. One ml of 50% polyethylene glycol in RMPI+0.01% EDTA was added to the cells and the cells were gently resuspended. After 1 minute, 1 ml of RPMI+0.01% EDTA was added without mixing. After 1 minute, another 4 ml of RPMI+0.01% EDTA was added without mixing. After 2 minutes, 4 ml of RPMI+20% fetal bovine serum (FBS) was added. Cells were pelleted by centrifugation at 50×g for 10 minutes. Cells were diluted with 30 ml of RPMI+20% FBS and the cells were resuspended. Fifteen ml of cell suspension was added to two 75 cm² collagen-coated tissue culture vessels. Cells adhered to the culture vessel overnight and were cultured for 1 week in RPMI+20% FBS with culture medium exchanged every other day. After 7 days, cells were examined by confocal microscopy for coexpression of TERT and albumin by immunohistochemistry, as described below. Unfused primary hepatocytes were non-viable after 7 days and did not contribute to resultant cell lines. If 100% of the cells were double positive for TERT and albumin, no further selection was performed. If less than 100% of the cells were double positive for TERT and albumin, cells were passaged by limiting dilution and re-tested for TERT and albumin until 100% of the cells were double positive. Of the 5 fusions performed, 2 fusions required no further selection, 3 fusions required only 2 passages before 100% expression of TERT and albumin. These cells were expanded by the method described in Example 4 and were used in the functional studies described herein.

### Example 4: Expansion of Fusion Cells

After stabilization of the fusion cells (after 1 week of culture in RPMI+20% FBS), cells were isolated from the culture vessel by incubation with Tryp-LE (Life Technologies, Grand Island, N.Y.). Cells were pelleted at 300×g for 5 minutes. Cells were resuspended at a concentration of 2×10⁴ cells/ml with the media formulation (Williams Medium E supplemented with 2% fatty acid-free BSA, 1% ITS solution, 5 mM hydrocortisone 21-hemisuccinate, FGF basic (20 ng/ml), FGF-4 (20 ng/ml), HFG (40 ng/ml), SCF (40 ng/ml), Oncostatin M(20 ng/ml), BMP-4 (20 ng/ml), EGF (40 ng/ml) and IL-1 beta (20 ng/ml), see Table 2). Cells were grown to confluence prior to harvesting for cryopreservation or further expansion. Those used in the study had undergone a minimum of 4 cycles of expansion, cryopreservation, and re-expansion with 5 PD per expansion cycle.

TABLE 2
Fusion Cell Expansion Medium

| | | |
|---|---|---|
| Williams Medium E | 500 | ml |
| Fatty acid-free Bovine Serum Albumin | 50 | ml |
| Glutamax | 5 | ml |
| Penicillin/Streptomycin | 5 | ml |
| Hydrocortisone-21- hemisuccinate | 5 | mM |
| ITS supplement | 5 | ml |
| Epithelial growth factor | 80 | ng/ml |
| Fibroblast growth factor basic | 20 | ng/ml |
| Fibroblast growth factor 4 | 20 | ng/ml |
| Hepatocyte growth factor | 40 | ng/ml |
| Stem cell factor | 40 | ng/ml |
| Oncostatin M | 20 | ng/ml |
| Bone morphogenic protein 4 | 20 | ng/ml |
| Interleukin 1 beta | 10 | ng/ml |

For karyotypic analysis of fusion cells, actively growing fusion cells were treated with colecemid overnight to arrest cells in metaphase. After, cells were harvested by standard cytogenetic protocol. They were treated with 0.75 MKCl hypotonic solution and fixed with 3:1 methanol:acetic acid. Cells were transferred to glass slides and stained with Wright's/Geimsa. Twenty G-banded metaphase cells were analyzed and 2 were karyotyped. Cells were analyzed using an Olympus BX61 microscope. The imaging and karyotyping of metaphase chromosomes was performed using Applied Spectral Imaging Software. Karyotypic analysis of the fusion cells showed no numerical or structural chromosomal abnormality associated with the over expression of TERT or the fusion process. The E12 MLPC cell line is from a male cord blood cell, the PH was from a female donor

### Example 5: Immunofluorescent Confocal Microscopy

Cells to be analyzed (control E12 TERT-MLPC, fusion cells from Examples 3 and 4, and primary hepatocytes (PH) from Zenotech) were harvested from their culture vessels by dissociation with Tryp-LE (Life Technologies, Grand Island, N.Y.). Cells were enumerated and resuspended in the medium described in Table 2, at a density of 10⁵ cells/ml. Two hundred μl of cell suspension was added to each well of a collagen-coated 16-well glass chamber slide (Nalge Nunc International, Rochester, N.Y.). Cells were cultured overnight to facilitate adherence to the slide. After attachment, cells were fixed for 1 hour in 1% formalin. After fixation, cells were permeabilized by PermaCyte Medium (CMDG, St Paul, Minn.). Cells were incubated with approximately 100 ng of antibodies (from R&D Systems (Minneapolis, Minn.) unless indicated otherwise) alkaline phosphatase (MAB1448), α-fetoprotein (MAB1369), albumin (MAB1456), c-reactive protein (MAB17071), hepatocyte growth factor receptor (MAB3583), nestin (MAB1269), SOX-17 (MAB1924), asialoglycoprotein receptor 1 (MAB4394), hepatocyte nuclear factor-4 (ABIN561308), GATA-4, α-1-antitrypsin, cytokeratin19 (MAB3608), SOX-2 (AF2018), SOX-9 (AF2018), EpCAM (MAB9601), Oct 3/4 (MAB1759), coagulation factor VII (MA5-16932), coagulation factor IX (HYB133-01-02) (from Invitrogen, Rockford, Ill.), P450 CYP 1A2 (ab151728), P450 CYP 3A4 (ab124921), glucuronosyltransferase isoforms UGT1A1 and UGT2B7 (ab126269 and ab194697 from Abcam, Cambridge, Mass.), and TERT (NB100-317 from Novus, Littleton, Colo.) for 40 minutes. Cells were then washed with PermaCyte to remove unbound antibody. Cells were then counter stained with secondary antibodies specific for mouse (A-11005), rabbit (A-11072), rat (A-11007) or goat (A-11080) antibody labelled with Alexa 594 dye (Life Technologies, Eugene, Oreg.). Cells were stained with DAPI dye to visualize the nucleus of each cell. Positivity of staining was confirmed by comparison to cells stained with antibody isotype controls. Comparative results of the confocal microscopy is shown in Table 3. Marker expression was confirmed by positive staining when compared to cells stained with antibody isotype controls (QTC1000, CMDG, St. Paul, Minn.) analyzed using the Olympus Fluoview 1000 confocal microscope. Comparative results of the confocal microscopy is shown in Table 3.

TABLE 3
Comparative Table of Hepatocyte-specific Marker Expression
Identified by Immunofluorescent Confocal Microscopy

| | E12- | | Primary Human |
|---|---|---|---|
| Analyte | MLPC | Fusion Cells | Hepatocytes |
| Alkaline Phosphatase | Neg | Pos | Pos |
| Alpha fetoprotein | Neg | Pos | Pos |
| Albumin | Neg | Pos | Pos |
| C-reactive protein | Neg | Pos | Pos |
| Hepatocyte Growth Factor Receptor | Neg | Pos | Pos |
| Coagulation Factor VII | Neg | Pos | Pos |
| Coagulation Factor IX | Neg | Pos | Pos |
| Nestin | Neg | Pos | Pos |
| SOX 17 | Neg | Pos | Pos |
| P450 CYP 3A4 | Neg | Pos | Pos |
| P450 CYP 1A2 | Neg | Pos | Pos |
| Asialo glycoprotein receptor 1 | Neg | Pos | Pos |
| Hepatocyte Nuclear Factor 4 | Neg | Cytoplasmic positive, | Cytoplasmic positive, |
| | | nuclear positive | nuclear positive |
| GATA-4 | Neg | Pos | Pos |
| Alpha-1-antitrypsin | Neg | Pos | Pos |
| SOX2 | Pos | Pos | Pos |
| SOX9 | Pos | Pos | Pos |
| CK19 | Pos | Pos | Pos |
| EpCAM | Pos | Pos | Pos |
| UGT1A1 | Neg | Pos | Pos |
| UGT2B7 | Neg | Pos | Pos |
| TERT | Pos | Pos | Weak in some cells |

Positive staining for the markers was demonstrated by detectable surface, cytoplasmic, or in the case of HNF4, nuclear staining, shown in red. Commitment to definitive endoderm is confirmed by positive staining for SOX17 and GATA-4. Further commitment to the hepatocyte phenotype was confirmed by other specific markers (see Table 3). Those of note were the positive expression of albumin, nuclear HNF4, the ASGP receptor, and cytochrome P450 isotypes 1A2 and 3A4 indicative of mature hepatocytes. The expression of glucuronosyltransferase isoforms UGT1A1 and UGT2B7, critical enzymes in liver that are essential for the conjugation and subsequent elimination of potentially toxic xenobiotic and endogenous compounds, including bilirubin, was demonstrated by immunohistochemistry in both the PH and the hybrid cells. In contrast, the native non-fused parental E12 cells showed no detectable expression.

### Example 6: Production of Albumin

Production of albumin is a critical and characteristic activity of hepatocytes. Albumin production by the different cell types (control E12 TERT-MLPC, fusion cells from Examples 3 and 4, and PH from Zenotech) was determined by the following procedure. Albumin production was analyzed with an enzymatic ELISA assay (Abcam, Cambridge, Mass.). Cells were cultured for 3 days in the medium specific for each cell type, after which they were dissociated from the culture plate using the Tryp-LE reagent, counted with hemocytometer and pelleted at 400×g for 5 minutes. Supernatant was discarded and cells were re-suspended in 1 ml of WIF water (Millipore/Sigma). Cells were then exposed to three repeated freeze-thaw cycles of freezing at −80° C. and thawing at room temperature to rupture and release the intracellular contents into the fluid phase. Cells were then centrifuged at 1000×g for 10 minutes to pellet the cell debris. Supernatant from the cell lysate was collected and analyzed by the albumin ELISA, according to the manufacturer's instructions. Results were standardized to 1×10⁶ cells/ml of supernatant to enable comparison between cell culture samples with different cell numbers. Resultant samples were analyzed on the Emax microplate reader (Molecular Devices, San Jose, Calif.) and processed with SoftMax PRO 4.8 Analysis Software. E12 MLPC and PHs were used as negative and positive controls, respectively. A total of four separate determinations were carried on different days. The results are shown in Table 4.

TABLE 4
Albumin and Urea Production

| Analyte | E12 | E12/PH Fusion | PH |
|---|---|---|---|
| Albumin production (pg/mL) | 0.7745 ± 0.92 | 5706.59 ± 4845.52 | 15725.38 ± 5490.89 |
| Urea (nmoles/mL) | 5.72 ± 5.61 | 78.475 ± 24.11 | 28.72 ± 22.9 |

### Example 7: Production of Urea

A critical function of hepatocytes is to convert toxic ammonia and uric acid to urea for excretion. Briefly, cells grown in collagen coated tissue culture vessels were dissociated from the culture vessel by incubation in TrypLE for 15 minutes. Cells were recovered and pelleted by centrifugation at 500×g for 5 minutes. Supernatant was removed and replaced with 1 ml of WFI (water for injection). Cells were frozen at −80° C. and thawed at room temperature 4 times to induce lysis of the cells. Lysates were centrifuged at 1000×g for 5 minutes to pellet remaining cell debris and the supernatant was analyzed for the presence of urea. Production of urea was analyzed by a standard colorimetric assay. The following cell populations were analyzed: TERT-MLPC/hepatocyte fusion cells with undifferentiated TERT-MLPC used as a negative control and primary human hepatocytes used as a positive control. The results of these experiments were standardized to 1×10⁶ cells and the results are shown in Table 4. E12 MLPCs produced very low levels of both urea and albumin when compared to primary hepatocytes. Fusion cells produced both urea and albumin levels that were comparable to the levels expressed by the primary human hepatocytes

### Example 8—FISH Analysis

Expression of the TERT gene in TERT-transfected cells was analyzed by fluorescent in situ hybridization using a QuantiGene ViewRNA probe set (Affimetrix, Santa Clara, Calif.). Briefly, MLPC-TERT cells were plated into 4 well chamber slides at a concentration of 1×10⁵ cells per well and allowed to adhere overnight. Using reagents included in the kit, cells were fixed with 1% Formalin for 1 hour, permeabilized with detergent for 15 minutes, treated with protease solution for 10 minutes to prepare for hybridization. Hybridization probe was added to sample and incubated for 3 hours at 40° C. Probe was removed by washing with PBS and the preamplifier mix was added and incubated for 30 minutes at 40° C. Pre-amplifier mix was removed by washing with PBS. Amplifier mix was added and incubated for 30 minutes at 40° C., after which amplifier mix was removed by washing with PBS. Label probe (Cy3 labeled) was added to cells and incubated for 30 minutes at 40° C. Excess label probe was removed by washing with PBS. Cells were counterstained with DAPI. Cells were analyzed by confocal microscopy on the Olympus Fluoview 1000 using the CY3 and DAPI filters.

E12 cells expressed high levels of TERT specific mRNA in 100% of the E12 cells. The presence of the gene product of TERT by antibody analysis in E12 cells was demonstrated by positive red staining in the cytoplasm of each cell. PH were shown to have low and sporadic expression of TERT by antibody analysis. High expression of TERT was observed in 100% of the E12/PH fusion cells by antibody analysis.

### Example 9—RT-PCR Analysis

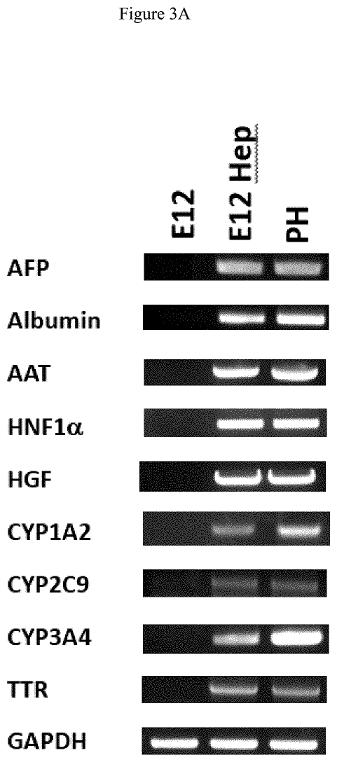

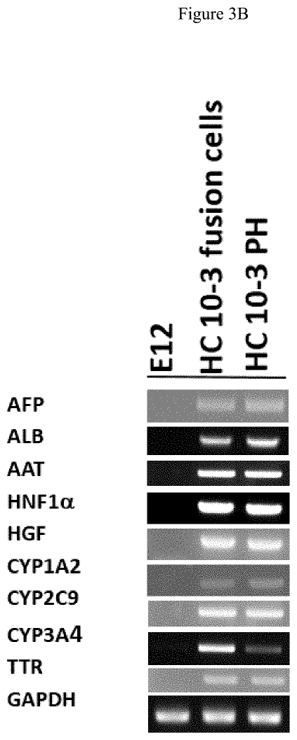

RNA isolates from E12 TERT-MLPC, primary hepatocytes (Zenotech, donor HC 10-3), E12 TERT-MLPC/PH HC10-3 fusion cells were analyzed by RT-PCR for liver-specific expression of hepatocyte-specific genes, including alpha-fetoprotein (AFP), α-1-antitrypsin (AAT), transthyretin (TTR), cytochrome P450 1A2 (CYP 1A2), cytochrome P450 3A4 (CYP 3A4), cytochrome P450 2C9 (CYP 2C9), hepatocyte nuclear factor 1α (HNF1A), hepatocyte growth factor (HGF), albumin (ALB), and housekeeping gene glyceraldehyde-3-phosphate dehydrogenase (GAPDH). Total RNA was extracted from the various cell populations using the UltraPure™ Phenol:Chloroform:Isoamyl Alcohol reagent (Invitrogen) and the Platinum® Quantitative RT-PCR ThermoScript™ One-Step System (Invitrogen) was used to carry out quantitative RT-PCR reactions according to manufacturer's recommendations. Total RNA isolated from E12 MLPC was used as the negative control, and that isolated from PH was used as a positive control. The primers for each of the hepatocyte-specific genes is shown in Table 5. Conditions for PCR reactions were initial denaturation at 94° C. for 3 min followed by 30 cycles of denaturation at 94° C. for 1 min, annealing for 1 min at 56° C., and elongation for 1 min at 72° C. PCR products were then resolved using a 1% agarose gel, and visualized under UV light. As shown in FIGS. 3A and 3B, both differentiated MLPC and fusion cells show similar levels of expression of hepatocyte-specific genes as PH.

TABLE 5
Primers used for RT-PCR Analysis

| Gene | | SEQ |
|---|---|---|
| Name | Primer Sequences | ID |
| AFP | F: 5′-TGCAGCCAAAGTGAAGAGGGAAGA-3 | 1 |
| | R: 5′-CATAGCGAGCAGCCCAAAGAAGAA-3′ | 2 |
| AAT | F: 5′-ACTGTCAACTTCGGGGACAC-3 | 3 |
| | R: 5′-CATGCCTAAACGCTTCATCA-3′ | 4 |
| TTR | F: 5′-TCATCGTCTGCTCCTCCTCT-3′ | 5 |
| | R: 5′-AGGTGICATCAGCAGCCTIT-3′ | 6 |
| CYP1A2 | F: 5′-CAATCAGGTGGTGGIGTCAG-3′ | 7 |
| | R: 5′-GCTCCTGGACTGTTFFCTGC-3′ | 8 |
| CYP3A4 | F: 5′-AAGTCGCCTCGAAGATACACA-3′ | 9 |
| | R: 5′-AAGGAGAGAACACTGCTCGTG-3′ | 10 |
| CYP2C9 | F: 5′-GGACAGAGACGACAAGCACA-3′ | 11 |
| | R: 5′-CATCTGTGTAGGGCATGIGG-3′ | 12 |
| HNF1A | F: 5′-TACACCACTCTGGCAGCCACACT-3′ | 13 |
| | R: 5′-CGGTGGGTACATTGGTGACAGAAC-3′ | 14 |
| GAPDH | F: 5′-GCACCGTCAAGGCTGAGAAC-3′ | 15 |
| | R: 5′-ATGGTGGTGAAGACGCCAGT-3′ | 16 |
| HGF | F: 5′-GTAAATGGGATTCCAACACGAACAA-3′ | 17 |
| | R: 5′-TGTFCGTGCAGTAAGAACCCAACTC-3′ | 18 |

The results described herein demonstrate the advantage of fusion between the immortalized MLPC and primary hepatocytes. E12 cells are negative for all hepatocyte-specific markers and positive for TERT. Hepatocytes are uniformly positive for hepatocyte-specific markers and mainly negative for TERT. The resultant fusion cells are uniformly positive for hepatocyte markers and TERT. This was further confirmed by PCR analysis where E12 MLPC were negative for all hepatocyte-specific RNA and the E12/HC10-3 fusion cells were uniformly positive for the hepatocytes-specific RNA as compared to human primary hepatocytes.

Functionality of resultant fusion cells was compared to the parental cell types, E12 MLPC and primary hepatocytes, by examining production of both urea and albumin. The E12 MLPC displayed very low levels of both urea and albumin production when compared to normal primary hepatocytes. In contrast, the E12/primary hepatocyte fusion cells produced significant levels of both urea and albumin, almost comparable for albumin, and even higher levels of urea when compared to primary hepatocytes.

The results provided herein a basis for the development of immortalized liver cell lines with large scale production capabilities for general drug metabolism and toxicology studies. In addition, the methods described herein can be employed to create immortalized hepatocyte-like cell lines with specific pathologies to provide a renewable source of cells for research and development of new therapies. Immortalized and potentially infinitely expandable populations of clonally-derived disease-specific hepatocytes can provide an important tool for the development of new therapies for those diseases.

The successful fusion of primary hepatocytes with an immortalized cord blood-derived stem cell line, such as the E12 cell line, resulting in a stable proliferative cell with the characteristics of hepatocytes can provide a procedural pathway for the development of other organ/cell-specific immortalized cell lines. Studies describing the in vitro differentiation of various stem cells to functional organ-specific cells (like hepatocytes) are characterized by large variances in the time to differentiation, growth factor compositions, viability and more. In vitro differentiation protocols suffer from the current inability to mimic all of the tissue-specific and time-dependent differentiation signals that occur during the development of the fetus. The ability to by-pass those complicated and time-consuming experiments necessary to clearly define the proper sequences of growth factors, culture conditions and the contributions of extra-cellular matrices can speed the development of organ and cell-specific immortalized cell lines. The application of a four decades-old simple and highly repeatable methodology that has been successfully applied to generate monoclonal antibodies could shortcut the development time for differentiated cells, and tissues. Employment of this methodology could enable similar cell fusions with other cell types creating immortalized cell lines with the characteristics of pancreas, neurons, and numerous other cell types.

Finally, a renewable source of functional hepatocyte-like cells could contribute significantly to the development of artificial liver support systems. One could imagine an extracorporeal system containing cells that would enable patients with liver failure to regenerate their own livers or survive as a bridge to transplant.

### Other Embodiments

The claims may suitably comprise, consist of, or consist essentially of, or be substantially free or free of any of the disclosed or recited elements. The claimed technology is illustratively disclosed herein can also be suitably practiced in the absence of any element which is not specifically disclosed herein. The various embodiments described above are provided by way of illustration only and should not be construed to limit the claims attached hereto. Various modifications and changes may be made without following the example embodiments and applications illustrated and described herein, and without departing from the true spirit and scope of the following claims.

What is claimed is:

1. A cell population comprising a plurality of hybrid cells, wherein each hybrid cell is composed of an immortalized multi-lineage progenitor cell (MLPC) and a primary somatic cell.

2. The cell population of claim 1, wherein said hybrid cell is created by the fusion of said immortalized MLPC and said primary somatic cell.

3. The cell population of claim 1, wherein said immortalized MLPC comprises a nucleic acid encoding a telomerase reverse transcriptase.

4. The cell population of claim 1, wherein said primary somatic cell is a hepatocyte.

5. The cell population of claim 1, wherein said hybrid cell has the biological activity associated with the primary somatic cell.

6. The cell population of claim 1, wherein the hybrid cell is immortalized and has the expandability of said immortalized MLPC.

7. The cell population of claim 1, wherein the hybrid cells can be expanded continuously in an expansion medium, said expansion medium comprising hydrocortisone, bovine serum albumin, insulin, transferrin, selenium, epithelial growth factor, basic fibroblast growth factor, fibroblast growth factor 4, hepatocyte growth factor, stem cell factor, oncostatin M, bone morphogenic protein 4, and interleukin 1 beta.

8. The cell population of claim 7, the expansion medium further comprising an antibiotic.

9. The cell population of claim 4, wherein said fusion cells are positive for alkaline phosphatase, alpha fetoprotein, albumin, c-reactive protein, hepatocyte growth factor receptor, complement factor VII, complement factor IX, nestin, SOX-17, P450 CYP 1A2, P450 CYP 3A4, asialo-glycoprotein receptor 1, hepatocyte nuclear factor-4, GATA-4, and alpha-1-antitrypsin.

10. The cell population of claim 1, said cells comprising a cryopreservative.

11. The cell population of claim 10, wherein said cryopreservative is fetal bovine serum, human serum, or human serum albumin in combination with one or more of the following: DMSO, trehalose, and dextran.

12. A method of producing hybrid cells, said method comprising a) combining immortalized multi-lineage progenitor cells and primary somatic cells in the presence of polyethylene glycol, b) culturing the mixture from step a) on a collagen-coated substrate; and c) selecting said hybrid cells that adhere to said substrate.

13. The method of claim 12, said method further comprising testing said hybrid cells for one or more characteristics of said primary somatic cells.

14. The method of claim 13, wherein said primary somatic cells are primary hepatocytes.

15. The method of claim 13, wherein said hybrid cells are tested for one or more of alkaline phosphatase, alpha fetoprotein, albumin, c-reactive protein, hepatocyte growth factor receptor, complement factor VII, complement factor IX, nestin, SOX-17, P450 CYP 1A2, P450 CYP 3A4, asialo-glycoprotein receptor 1, hepatocyte nuclear factor-4, GATA-4, and alpha-1-antitrypsin.

16. The method of claim 14, wherein said hybrid cells are tested for albumin production.

17. The method of claim 14, wherein said hybrid cells are tested for urea production.

18. A method of producing an expanded population of hybrid cells, the method comprising:
a) providing a collagen-coated culturing device housing a purified population of hybrid cells, wherein each hybrid cell is composed of an immortalized MLPC and a primary hepatocyte, and
b) culturing the hybrid cell in an expansion medium.

19. The method of claim 18, wherein said expansion medium comprises hydrocortisone, bovine serum albumin, insulin, transferrin, selenium, epithelial growth factor, basic fibroblast growth factor, fibroblast growth factor 4, hepatocyte growth factor, stem cell factor, oncostatin M, bone morphogenic protein 4, and interleukin 1 beta.

20. The method of claim 18, said method further comprising testing said hybrid cells for alkaline phosphatase, alpha fetoprotein, albumin, c-reactive protein, hepatocyte growth factor receptor, complement factor VII, complement factor IX, nestin, SOX-17, P450 CYP 1A2, P450 CYP 3A4, asialo-glycoprotein receptor 1, hepatocyte nuclear factor-4, GATA-4 and alpha-1-antitrypsin.

21. An article of manufacture comprising the population of cells of claim 1.

22. An article of manufacture of claim 21, wherein said population is housed within a container.

23. An article of manufacture of claim 22, wherein said container is a vial, bottle, or a bag.

24. An article of manufacture of claim 23, wherein said container further comprises a cryopreservative.
